(19) United States
(12) Patent Application Publication
(10) Pub. No.: US 2004/0152650 A1
(43) Pub. Date: Aug. 5, 2004

(54) Preventing ischemia-induced cell damage

(76) Inventor: Keith A. Webster, Key Biscayne, FL (US)

Correspondence Address: Akerman Senterfitt, Suite 400, 222 Lakeview Avenue, West Palm Beach, FL, 33401-6183, US

(21) Appl. No.: 10/624,880

(22) Filed: Jul. 22, 2003

Related U.S. Application Data
(60) Provisional application No. 60/397,841, filed on Jul. 22, 2002.

(51) Int. Cl. A61K 48/00; C12N 5/02

(52) U.S. Cl. 514/44; 435/375

(57) ABSTRACT

Hypoxia-acidosis-associated cell death is mediated by BNIP3, a member of the Bcl-2 family of apoptosis-regulating proteins. Chronic hypoxia induced the expression and accumulation of BNIP3 mRNA and protein in cardiac myocytes but acidosis was required to activate the death pathway. Acidosis stabilized BNIP3 protein and increased the association with mitochondria. Cell death by hypoxia-acidosis was blocked by pretreatment with antisense BNIP3 oligonucleotides. The pathway included extensive DNA fragmentation and opening of the mitochondrial permeability transition pore but no apparent caspase activation. Overexpression of wild type BNIP3, but not a translocation-defective mutant activated cardiac myocyte death when the myocytes were acidotic or hypoxic.

### CROSS-REFERENCE TO RELATED APPLICATION

The present application claims the priority of U.S. provisional patent application serial No. 60/397,841 filed Jul. 22, 2002.

### FIELD OF THE INVENTION

The invention relates generally to the fields of biology, medicine, and pathophysiology. More particularly, the invention relates to methods and compositions for preventing cellular damage and/or death caused by ischemia.

### BACKGROUND

Atherosclerotic plaque restricts blood flow through coronary arteries and provides a substrate for occlusive thrombus formation. Reduced blood flow produces hypoxia in the tissues downstream of the lesion; complete occlusion leads to severe hypoxia that threatens the viability of the myocardium. Subsequent reperfusion by thrombolysis or removal of the plaque may subject cells to further damage through oxidative stress, and a region of permanent injury containing dead and dying cells develops (the “infarct”) (1). Hypoxia may persist within the infarct and at its margins for days or weeks, exacerbating the injury (2-4). In response, the ischemic myocardium switches from respiration to glycolytic energy metabolism, with increased glucose consumption, lactic acid production, and lower intracellular pH (5-7). The extent of tissue loss to infarction is determined by the severity and duration of the ischemic period and is known to involve both necrotic and apoptotic cell death pathways (8-10). Oxidative stress caused by reperfusion may account for 50% of the tissue damage during early infarction (11-13). Multiple additional factors contribute to cell death as the infarcted area expands and more border cells die. These include collateral damage from necrosis and infiltrating macrophages (14), additional necrotic death resulting from energy depletion (15-18), and changes associated with hypoxia.

BNIP3 is a member of the so-called BH3-only subfamily of Bcl-2-family proteins that heterodimerize and antagonize the activity of pro-survival proteins (Bcl-2, Bcl-XL) and promote apoptosis (19;20). Proteins in this group do not possess the same Bcl-2 homology domains (BH1 and BH2) as the other Bcl-2 family members but may bind through a common BH3 domain. Bcl-2 proteins are usually associated with cell membranes, particularly the mitochondria, where they are anchored by a COOH-terminal domain. Individual family members may remain in the cytosol or be loosely membrane bound and translocate into membranes only after a death signal is received (19;21;22). A major function of this class of proteins is to determine the on/off state of the mitochondrial permeability transition pore (MPTP) (23-25). Although it contains a partial BH3 domain, the C-terminal transmembrane domain of BNIP3 is essential for membrane targeting and promotion of apoptosis. BNIP3 expression is normally undetectable in most organs, including the heart, but can be induced by hypoxia (19). Overexpression of BNIP3 protein by transfection of the cDNA into some cultured cell lines results in membrane translocation and initiation of a cell death pathway with features similar to necrosis (19).

The role of hypoxia in ischemia-mediated death of cardiac cells is controversial. It was recently shown that hypoxia alone is not a major stimulus for apoptosis, and that significant cell death requires the combination of hypoxia and acidosis (26). Acidosis regularly accompanies ischemia because of increased accumulation of lactic and phosphoric acid. Acidosis has previously been implicated in cardiac cell death: inhibition of the vacuolar ATPase, a proton pump involved in pH regulation, promotes apoptosis of cardiac myocytes

### SUMMARY

The invention relates to the discovery that BNIP3 is a molecular effector of hypoxia acidosis-mediated apoptosis. In particular, it was found that: (1) irreversible damage to cardiac myocytes caused by hypoxia and acidosis during myocardial ischemia is mediated exclusively by BNIP3 and can be blocked by BNIP3 antisense treatment; (2) BNIP3 is induced by hypoxia but remains inactive until the intracellular pH decreases; and (3) transfection of mutant BNIP3 cDNAs inhibits the activation of BNIP3 by acidosis and blocks cell death. Although BNIP3 was previously known to be pro-apoptotic and inducible by hypoxia, it was a surprising finding that exogenously introduced BNIP3 mutant proteins would block the activation of endogenous BNIP3 by acidosis and protect heart cells against ischemic damage.

Accordingly, the invention features a method for preventing or reducing hypoxia-acidosis induced injury to a cell. This method includes the step of reducing BNIP3 expression or activity in the cell. In this method, the step of reducing BNIP3 expression or activity in the cell can include decreasing the amount of BNIP3 mRNA or BNIP3 protein in the cell. Reducing the amount of BNIP3 mRNA can be performed by introducing an antisense oligonucleotide into the cell. Reducing BNIP3 activation in the cell can include expressing a mutant BNIP3 protein in the cell, preventing BNIP3 protein dimerization in the cell, and/or preventing translocation of BNIP3 protein to a mitochondrion in the cell. The latter can be accomplished by contacting the cell with a small peptide (e.g., one 5-30 amino acid residues in length) or a non-protein non-peptide organic molecule. This can also be achieved by contacting the cell with a viral vector, e.g., one derived from a virus such as an adenovirus, an adenoviral associated virus (AAV), or a lentivirus. The step of reducing BNIP3 expression or activity in the cell can also include preventing or reversing acidosis in the cell.

Among the cells that can be targeted by methods of the invention include myocytes such as cardiomyocytes or skeletal muscle cells, neurons, hepatocytes, kidney cells, eye cells, bone marrow cells, and lung cells. These can be within an animal such as a human subject.

In another aspect the invention features a method for upregulating or inducing injury in a cell. This method includes the step of upregulating BNIP3 expression or activity in the cell. In this method, the cell can be a tumor cell such as one within an animal, e.g., a human subject. Also within the invention are compositions for modulating BNIP3 expression or activity in a cell. These compositions include an agent that can upregulate or down-regulate BNIP3 expression or activity in the cell. Examples of such an agent include sense and antisense BNIP3 nucleic acids, including those incorporated in vectors such as viral vectors; native and mutant BNIP3 proteins and non-peptide analogues thereof; and small molecule agonists and antagonists of BNIP3 activity (e.g., organic and inorganic molecules).

As used herein, the term “gene” means a nucleic acid molecule that codes for a particular protein, or in certain cases, a functional or structural RNA molecule. For example, a BNIP3 gene encodes a BNIP3 protein. The phrase “nucleic acid” or a “nucleic acid molecule” means a chain of two or more nucleotides such as RNA (ribonucleic acid) and DNA (deoxyribonucleic acid). A “purified” nucleic acid molecule is one that is substantially separated from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

The phrases “BNIP3 gene,” “BNIP3 polynucleotide,” or “BNIP3 nucleic acid” as used herein mean a native BNIP3-encoding nucleic acid sequence, e.g., the native human (NLM Accession No. NM_{—}004052) BNIP3 mRNA; a native form BNIP3 cDNA; a nucleic acid having sequences from which a BNIP3 cDNA can be transcribed; and/or allelic variants and homologs of the foregoing. The terms encompass double-stranded DNA, single-stranded DNA, and RNA.

As used herein, “protein” or “polypeptide” mean any peptide-linked chain of amino acids, regardless of length or post-translational modification, e.g., glycosylation or phosphorylation. A “purified” polypeptide is one that is substantially separated from other polypeptides in a cell or organism in which the polypeptide naturally occurs (e.g., 30, 40, 50, 60, 70, 80, 90, 95, 96, 97, 98, 99, 100% free of contaminants).

By the phrase “BNIP3 protein” or “BNIP3 polypeptide” is meant an expression product of a BNIP3 gene such as a native BNIP3 protein (for human BNIP3 see Swissprot Accession No. Q12983), or a protein that shares at least 65% (but preferably 75, 80, 85, 90, 95, 96, 97, 98, or 99%) amino acid sequence identity with one of the foregoing and displays a functional activity of a human native BNIP3 protein. A “functional activity” of a protein is any activity associated with the physiological function of the protein. For example, functional activities of a native BNIP3 protein may include dimerization with another BNIP3 protein, translocation to a mitochondrion, and regulation of hypoxia-acidosis induced cell damage.

When referring to a nucleic acid molecule or polypeptide, the term “native” refers to a naturally-occurring (e.g., a “wild-type”) nucleic acid or polypeptide. A “homolog” of a BNIP3 gene from one species of organism is a gene sequence encoding a BNIP3 polypeptide isolated from an organism of a different species. Similarly, a “homolog” of a native BNIP3 polypeptide is an expression product of a BNIP3 gene homolog.

A “fragment” of a BNIP3 nucleic acid is a portion of a BNIP3 nucleic acid that is less than full-length and comprises at least a minimum length capable of hybridizing specifically with a native BNIP3 nucleic acid under stringent hybridization conditions. The length of such a fragment is preferably at least 15 nucleotides, more preferably at least 20 nucleotides, and most preferably at least 30 nucleotides of a native BNIP3 nucleic acid sequence. A “fragment” of a BNIP3 polypeptide is a portion of a BNIP3 polypeptide that is less than full-length (e.g., a polypeptide consisting of 5, 10, 15, 20, 30, 40, 50, 75, 100 or more amino acids of a native BNIP3 protein), and preferably retains at least one functional activity of a native BNIP3 protein.

Unless otherwise defined, all technical terms used herein have the same meaning as commonly understood by one of ordinary skill in the art to which this invention belongs. All publications, patent applications, patents, and other references mentioned herein are incorporated by reference in their entirety. In the case of conflict, the present specification, including definitions will control.

### BRIEF DESCRIPTION OF THE DRAWINGS

FIG. 1. BNIP3 induction correlates with increased apoptosis. (A) Cardiac myocytes were subjected to hypoxia with (left panel) or without (right panel) medium change, harvested at the indicated times and processed for genomic fragmentation assays. The pH of the media at the time of harvesting is shown at bottom. (B). Northern blots of cardiac myocyte RNA extracted from hypoxic cultures. (C). Western blot analysis of proteins from hypoxic cardiac myocytes as in (A). Anti-BNIP3 recognizes 2 bands at approximately 60 kD and 30 kD, corresponding to SDS-resistant homodimers and monomers respectively. Lower panels show the same blot probed with anti-Bax, Bak, and β-Actin respectively. Results are representative of at least 3 experiments.

FIG. 2. BNIP3 antisense inhibits programmed death of cardiac myocytes. (A) Cultures were incubated with BNIP3 (AS) or random sequence (R) oligonucleotides as described below, subjected to hypoxia-acidosis as indicated and analyzed for DNA fragmentation. (B) Cardiac myocytes were treated with oligonucleotides as in (A) and extracted proteins were analyzed by western blots with anti-BNIP3 or β-actin. Results are representative of 2 experiments.

FIG. 3. Association of BNIP3 with subcellular fractions. Cardiac myocytes were subjected to hypoxia as described in FIG. 1. At the indicated times cells were harvested, rinsed, lysed and subjected to alkaline solubilization (right panels) as described in Methods (see Example 1 below). After treatments, samples were separated into subcellular fractions and analyzed by Western blots. Blots were stripped and re-probed with anti-succinate dehydrogenase (Upstate Biotechnology, NY) probes to define the purity of fractions. Results are representative of 2 separate experiments.

FIG. 4. Characteristic of programmed death by BNIP3. (A) Cardiac myocytes were subjected to hypoxia-acidosis as described in FIG. 1. At the indicated times samples of media were taken for analysis of LDH activity (open circles) or plates were stained with trypan blue (closed circles). Data is expressed as % of cells stained with trypan blue or % LDH released relative to total LDH in homogenates. (B) Cardiac myocytes were subjected to hypoxia-acidosis in the absence or presence of the broad-range caspase inhibitor Boc-D as indicated. Staurosporine (Sta; 0.1 μM for 8 h) is shown as a positive control. (C) Cardiac myocytes were subjected to hypoxia-acidosis in the absence or presence of the MPTP inhibitors bongrekic acid (BA) or decylubiquinone (DUB), as indicated. (D). Cardiac myocytes were exposed to normoxic or hypoxia-acidosis conditions. At the times indicated cells were loaded with MitoTracker Red dye and analyzed by confocal microscopy. Arrows indicate intense staining around nuclei in aerobic myocytes. Results are representative of 3 experiments.

FIG. 5. Programmed death of BNIP3-transfected cardiac myocytes. Cardiac myocytes were transfected with expression plasmids containing β-Gal plus empty vector, β-Gal with BNIP3 or β-Gal with BNIP3-deltaTM, as indicated. After 48 h, transfected cultures were exposed to continued normoxic culture or to hypoxia, acidosis, or hypoxia +acidosis as described in Methods. At the indicated times, plates were rinsed, co-stained with X-gal and Hoechst 33342 and visualized by microscopy. Bars indicate SEM from at least 200 X-gal positive cells per condition. * (p<0.002) and ** (p<0.001) refer to the respective condition compared with aerobic controls. BNIP3delta-TM transfection with 8 h hypoxia +acid was significantly different from either β-Gal or BNIP3 8 h hypoxia +acid (p<0.05 and 0.01 respectively).

FIG. 6. Acid-mediated opening of MPTP in BNP3-transfected myocytes. Cardiac myocytes were co-transfected with pGFP and BNIP3-wt or BNIP3delta-TM, as indicated. After 48 h, transfected cultures were exposed to continued culture at neutral pH or to acidosis for an additional 8 h as described below in Methods. Cultures were stained with Mitotracker Red dye, fixed, and analyzed as described in the description of FIG. 4. At least 50 GFP-positive monocytes per condition were scored from 4 separate dishes.

FIG. 7. Protection against BNIP-3-apoptosis by a TAT-peptide containing an N-terminal fragment of BNIP3. A shows DNA fragmentation gels at 24 and 48 h. B is a graph showing percent cell death.

### DETAILED DESCRIPTION

The invention provides compositions and methods for modulating BNIP3-induced cell death. These compositions and method can be used to prevent or reduce expression or activity of BNIP3 in cells. For example, BNIP3 expression can be prevented or reduced in a cell by inhibiting production of BNIP3 mRNA or protein, by preventing or reversing dimerization of BNIP3 proteins, or by preventing or reversing translocation of BNIP3 protein to a mitochondrion. The below described preferred embodiments illustrate adaptations of these compositions and methods. Nonetheless, from the description of these embodiments, other aspects of the invention can be made and/or practiced.

### Biological Methods

Methods involving conventional molecular biology techniques are described herein. Such techniques are generally known in the art and are described in detail in methodology treatises such as Molecular Cloning: A Laboratory Manual, 2 nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, 81991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Modulating BNIP3 Expression of Activity in a Cell

The invention provides methods and compositions for modulating BNIP3 expression and/or activity in a cell. Numerous agents for modulating expression/activty of intracellular proteins such as BNIP3 in a cell or known. Any of these suitable for the particular system being used may be employed. Typical agents for modulating expression of intracellular proteins are mutants proteins, nucleic acids, and small organic or inorganic molecules.

Examples of proteins that can modulate BNIP3 expression and/or activity in a cell include native BNIP3 proteins (e.g., to upregulate activity) or variants thereof that can compete with a native BNIP3 protein for binding ligands such as another BNIP3 protein (e.g., to downregulate activity). Such protein variants can be generated through various techniques known in the art. For example, BNIP3 protein variants can be made by mutagenesis, such as by introducing discrete point mutation(s), or by truncation (e.g., of the transmembrane region). Mutation can give rise to a BNIP3 protein variant having substantially the same, or merely a subset of the functional activity of a native BNIP3 protein. Alternatively, antagonistic forms of the protein can be generated which are able to inhibit the function of the naturally occurring form of the protein, such as by competitively binding to another molecule that interacts with BNIP3 protein. In addition, agonistic (or superagonistic) forms of the protein may be generated that constitutively express on or more BNIP3 functional activities. Other variants of BNIP3 proteins that can be generated include those that are resistant to proteolytic cleavage, as for example, due to mutations which alter protease target sequences. Whether a change in the amino acid sequence of a peptide results in a BNIP3 protein variant having one or more functional activities of a native BNIP3 protein can be readily determined by testing the variant for a native BNIP3 protein functional activity (e.g., modulating a cellular response).

Another agent that can modulate BNIP3 expression/activity is a BNIP3 non-peptide mimetic or chemically modified form of BNIP3 that disrupts binding of a BNIP3 protein to other proteins or molecules with which the native BNIP3 protein interacts. See, e.g., Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopepitides (Ewenson et al. (1986) J. Med. Chem. 29:295; and Ewenson et al. in Peptides: Structure and Function (Proceedings of the 9th American Peptide Symposium) Pierce Chemical Co. Rockland, Ill, 1985), beta-turn dipeptide cores (Nagai et al. (1985) Tetrahedron Lett 26:647; and Sato et al. (1986) J. Chem. Soc. Perkin. Trans. 1:1231), and beta-aminoalcohols (Gordon et al. (1985) Biochem. Biophys. Res. Commun. 126:419; and Dann et al. (1986) Biochem. Biophys. Res. Commun. 134:71). BNIP3 proteins may also be chemically modified to create BNIP3 protein derivatives by forming covalent or aggregate conjugates with other chemical moieties, such as glycosyl groups, lipids, phosphate, acetyl groups and the like. Covalent derivatives of BNIP3 protein can be prepared by linking the chemical moieties to functional groups on amino acid side chains of the protein or at the N-terminus or at the C-terminus of the polypeptide.

The agent that directly reduces expression/activity of BNIP3 can also be a nucleic acid that modulates expression of BNIP3. For example, the nucleic acid can be a sense nucleic acid that encodes a BNIP3 protein (e.g., introduction into a cell can increase the cells BNIP3 activity). The nucleic acid can also be an antisense nucleic acid that hybridizes to mRNA encoding BNIP3. Antisense nucleic acid molecules for use within the invention are those that specifically hybridize (e.g. bind) under cellular conditions to cellular mRNA and/or genomic DNA encoding a BNIP3 protein in a manner that inhibits expression of the BNIP3 protein, e.g., by inhibiting transcription and/or translation. The binding may be by conventional base pair complementarity, or, for example, in the case of binding to DNA duplexes, through specific interactions in the major groove of the double helix.

Antisense constructs can be delivered as an expression plasmid which, when transcribed in the cell, produces RNA which is complementary to at least a unique portion of the cellular mRNA which encodes a BNIP3 protein. Alternatively, the antisense construct can take the form of an oligonucleotide probe generated ex vivo which, when introduced into a BNIP3 protein expressing cell, causes inhibition of BNIP3 protein expression by hybridizing with an mRNA and/or genomic sequences coding for BNIP3 protein. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al., Biotechniques 6:958-976, 1988; and Stein et al., Cancer Res. 48:2659-2668, 1988. With respect to antisense DNA, oligodeoxyribonucleotides derived from the translation initiation site, e.g., between the −10 and +10 regions of a BNIP3 protein encoding nucleotide sequence, are preferred.

Antisense approaches involve the design of oligonucleotides (either DNA or RNA) that are complementary to BNIP3 mRNA. The antisense oligonucleotides will bind to BNIP3 mRNA transcripts and prevent translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R., Nature 372:333, 1994). Therefore, oligonucleotides complementary to either the 5′ or 3′ untranslated, non-coding regions of a BNIP3 gene could be used in an antisense approach to inhibit translation of endogenous BNIP3 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′, 3′ or coding region of BNIP3 mRNA, antisense nucleic acids should be at least eighteen nucleotides in length, and are preferably less than about 100 and more preferably less than about 30, 25, 20, or 18 nucleotides in length.

Antisense oligonucleotides of the invention may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxyethyl) uracil, 5-carboxymethylaminomethyl-2-thiouridine, 5-carboxymethylaminomethyluracil, dihydrouricil, beta-D-galactosylqueosine, inosine, N6-isopentenyladenine, 1-methylguanine, 1-methylinosine, 2,2-idimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Antisense oligonucleotides of the invention may also comprise at least one modified sugar moiety selected from the group including but not limited to arabinose, 2-fluoroarabinose, xylulose, and hexose; and may additionally include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids Res. 15:6625-6641, 1987). Such oligonucleotide can be a 2′-O-methylribonucleotide (Inoue et al., Nucl. Acids Res. 15:6131-6148, 1987), or a chimeric RNA-DNA analogue (Inoue et al., FEBS Lett. 215:327-330, 1987).

Oligonucleotides of the invention may be synthesized by standard methods known in the art, e.g by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209), methylphosphonate oligonucleotides can be prepared by use of controlled pore glass polymer supports (Sarin et al., Proc. Natl. Acad. Sci. U.S.A. 85:7448-7451, 1988).

The nucleic acid molecules should be delivered into cells that express BNIP3 in vivo. A number of methods have been developed for delivering DNA or RNA into cells. For instance, such molecules can be introduced directly into the tissue site by such standard techniques as electroporation, liposome-mediated transfection, CaCl-mediated transfection, or the use of a gene gun. Alternatively, modified antisense molecules, designed to target the desired cells (e.g., antisense linked to peptides or antibodies that specifically bind receptors or antigens expressed on the target cell surface) can be used.

Because it is often difficult to achieve intracellular concentrations of the antisense sufficient to suppress translation of endogenous mRNAs, a preferred approach utilizes a recombinant DNA construct in which the antisense oligonucleotide is placed under the control of a strong promoter (e.g., the CMV promoter). The use of such a construct to transform cells will result in the transcription of sufficient amounts of single stranded RNAs that will form complementary base pairs with the endogenous BNIP3 transcripts and thereby prevent translation of BNIP3 mRNA.

Ribozyme molecules designed to catalytically cleave BNIP3 mRNA transcripts can also be used to prevent translation of BNIP3 mRNA and expression of BNIP3 protein (see, e.g., PCT Publication No. WO 90/11364, published Oct. 4, 1990; Sarver et al., Science 247:1222-1225, 1990 and U.S. Pat. No. 5,093,246). While ribozymes that cleave mRNA at site specific recognition sequences can be used to destroy BNIP3 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591, 1988. Preferably the ribozyme is engineered so that the cleavage recognition site is located near the 5′ end of BNIP3 mRNA; i.e., to increase efficiency and minimize the intracellular accumulation of non-functional mRNA transcripts. Ribozymes within the invention can be delivered to a cell using a vector.

Endogenous BNIP3 gene expression can also be reduced by inactivating or “knocking out” the BNIP3 gene or its promoter using targeted homologous recombination. See, e.g, Kempin et al., Nature 389: 802 (1997); Smithies et al., Nature 317:230-234, 1985; Thomas and Capecchi, Cell 51:503-512, 1987; and Thompson et al., Cell 5:313-321, 1989. For example, a mutant, non-functional BNIP3 gene variant (or a completely unrelated DNA sequence) flanked by DNA homologous to the endogenous BNIP3 gene (either the coding regions or regulatory regions of the BNIP3 gene) can be used, with or without a selectable marker and/or a negative selectable marker, to transfect cells that express BNIP3 protein in vivo.

Alternatively, endogenous BNIP3 gene expression might be reduced by targeting deoxyribonucleotide sequences complementary to the regulatory region of the BNIP3 gene (i.e., the BNIP3 promoter and/or enhancers) to form triple helical structures that prevent transcription of the BNIP3 gene in target cells. (See generally, Helene, C., Anticancer Drug Des. 6(6):569-84, 1991; Helene, C., et al., Ann. N.Y. Acad. Sci. 660:27-36, 1992; and Maher, L. J., Bioassays 14(12):807-15, 1992). Inhibition of BNIP3 gene expression might also be performed using RNA interference (RNAi) techniques.

The nucleic acids, ribozyme, and triple helix molecules used in the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

### BNIP3 Mutants

Methods of the present invention may utilize a purified BNIP3 protein encoded by a nucleic acid of the invention. A preferred form of BNIP3 is a purified native human BNIP3 protein that has the amino acid sequence deposited with SwissProt as Accession No. Q12983.

Variants of native BNIP3 proteins such as fragments, analogs and derivatives of native BNIP3 proteins may also be used in methods of the invention. Such variants include, e.g., a polypeptide encoded by a naturally occurring allelic variant of a native BNIP3 gene, a polypeptide encoded by an alternative splice form of a native BNIP3 gene, a polypeptide encoded by a homolog of a native BNIP3 gene, and a polypeptide encoded by a non-naturally occurring variant of a native BNIP3 gene.

BNIP3 protein variants have a peptide sequence that differs from a native BNIP3 protein in one or more amino acids. The peptide sequence of such variants can feature a deletion, addition, or substitution of one or more amino acids of a native BNIP3 polypeptide. Amino acid insertions are preferably of about 1 to 4 contiguous amino acids, and deletions are preferably of about 1 to 10 contiguous amino acids. In some applications, variant BNIP3 proteins substantially maintain a native BNIP3 protein functional activity (e.g., ability to mediate hypoxia-acidosis related cell damage). For other applications, variant BNIP3 proteins lack or feature a significant reduction in a BNIP3 protein functional activity. Where it is desired to retain a functional activity of native BNIP3 protein, preferred BNIP3 protein variants can be made by expressing nucleic acid molecules within the invention that feature silent or conservative changes. Variant BNIP3 proteins with substantial changes in functional activity can be made by expressing nucleic acid molecules within the invention that feature less than conservative changes.

BNIP3 protein fragments corresponding to one or more particular motifs and/or domains or to arbitrary sizes, for example, at least 5, 10, 25, 50, 75, 100, 125, 150, or 175 amino acids in length may be utilized in methods of the present invention. Isolated peptidyl portions of BNIP3 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a BNIP3 protein used in methods of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a native BNIP3 protein.

Methods of the invention may also involve recombinant forms of the BNIP3 proteins. Recombinant polypeptides preferred by the present invention, in addition to native BNIP3 protein, are encoded by a nucleic acid that has at least 85% sequence identity (e.g., 85, 86, 87, 88, 89, 90, 91, 92, 93, 94, 95, 96, 97, 98, 99, 100%) with a native BNIP3 nucleic acid sequence. In a preferred embodiment, variant BNIP3 proteins lack one or more functional activities of a native BNIP3 protein.

BNIP3 protein variants can be generated through various techniques known in the art. For example, BNIP3 protein variants can be made by mutagenesis, such as by introducing discrete point mutation(s), or by truncation. Mutation can give rise to a BNIP3 protein variant having substantially the same, or merely a subset of the functional activity of a native BNIP3 protein. Alternatively, antagonistic forms of the protein can be generated which are able to inhibit the function of the naturally occurring form of the protein, such as by competitively binding to another molecule that interacts with BNIP3 protein. In addition, agonistic forms of the protein may be generated that constitutively express one or more BNIP3 functional activities. Other variants of BNIP3 proteins that can be generated include those that are resistant to proteolytic cleavage, as for example, due to mutations that alter protease target sequences. Whether a change in the amino acid sequence of a peptide results in a BNIP3 protein variant having one or more functional activities of a native BNIP3 protein can be readily determined by testing the variant for a native BNIP3 protein functional activity.

Nucleic acid molecules encoding BNIP3 fusion proteins may be used in methods of the invention. Such nucleic acids can be made by preparing a construct (e.g., an expression vector) that expresses a BNIP3 fusion protein when introduced into a suitable host. For example, such a construct can be made by ligating a first polynucleotide encoding a BNIP3 protein fused in frame with a second polynucleotide encoding another protein such that expression of the construct in a suitable expression system yields a fusion protein.

As another example, BNIP3 protein variants can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential BNIP3 protein sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) Proc. Natl. Acad. Sci. USA 89:2429-2433; Devlin et al. (1990) Science 249: 404-406; Cwirla et al. (1990) Proc. Natl. Acad. Sci. USA 87: 6378-6382; as well as U.S. Pat. Nos. 5,223,409; 5,198,346; and 5,096,815).

Similarly, a library of coding sequence fragments can be provided for a BNIP3 gene clone in order to generate a variegated population of BNIP3 protein fragments for screening and subsequent selection of fragments having one or more native BNIP3 protein functional activities. A variety of techniques are known in the art for generating such libraries, including chemical synthesis. In one embodiment, a library of coding sequence fragments can be generated by (i) treating a double-stranded PCR fragment of a BNIP3 gene coding sequence with a nuclease under conditions wherein nicking occurs only about once per molecule; (ii) denaturing the double-stranded DNA; (iii) renaturing the DNA to form double-stranded DNA which can include sense/antisense pairs from different nicked products; (iv) removing single-stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of BNIP3 gene variants. The most widely used techniques for screening large gene libraries typically involve cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Combinatorial mutagenesis has a potential to generate very large libraries of mutant proteins, e.g., in the order of 1026 molecules. To screen a large number of protein mutants, techniques that allow one to avoid the very high proportion of non-functional proteins in a random library and simply enhance the frequency of functional proteins (thus decreasing the complexity required to achieve a useful sampling of sequence space) can be used. For example, recursive ensemble mutagenesis (REM), an algorithm that enhances the frequency of functional mutants in a library when an appropriate selection or screening method is employed, might be used. Arkin and Yourvan (1992) Proc. Natl. Acad. Sci. USA 89:7811-7815; Yourvan et al. (1992) Parallel Problem Solving from Nature, 2., In Maenner and Manderick, eds., Elsevier Publishing Co., Amsterdam, pp. 401-410; Delgrave et al. (1993) Protein Engineering 6(3):327-331.

Methods of the invention may utilize mimetics, e.g. peptide or non-peptide agents, that are able to disrupt binding of a BNIP3 protein to other proteins or molecules with which a native BNIP3 protein interacts. Thus, the mutagenic techniques described herein can also be used to map which determinants of BNIP3 protein participate in the intermolecular interactions involved in, for example, binding of a BNIP3 protein to other proteins which may function upstream (e.g., activators or repressors of BNIP3 functional activity) of the BNIP3 protein or to proteins or nucleic acids which may function downstream of the BNIP3 protein, and whether such molecules are positively or negatively regulated by the BNIP3 protein. To illustrate, the critical residues of a BNIP3 protein which are involved in molecular recognition of, for example, the BNIP3 protein or other components upstream or downstream of the BNIP3 protein can be determined and used to generate BNIP3 protein-derived peptidomimetics which competitively inhibit binding of the BNIP3 protein to that moiety. By employing scanning mutagenesis to map the amino acid residues of a BNIP3 protein that are involved in binding other proteins, peptidomimetic compounds can be generated which mimic those residues of a native BNIP3 protein. Such mimetics may then be used to interfere with the normal function of a BNIP3 protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopepitides (Ewenson et al. (1986) J. Med. Chem. 29:295; and Ewenson et al. in Peptides: Structure and Function (Proceedings of the 9th American Peptide Symposium) Pierce Chemical Co. Rockland, Ill, 1985), eta-turn dipeptide cores (Nagai et al. (1985) Tetrahedron Lett 26:647; and Sato et al. (1986) J. Chem. Soc. Perkin. Trans. 1:1231), and beta-aminoalcohols (Gordon et al. (1985) Biochem. Biophys. Res. Commun. 126:419; and Dann et al. (1986) Biochem. Biophys. Res. Commun. 134:71). BNIP3 proteins may also be chemically modified to create BNIP3 protein derivatives by forming covalent or aggregate conjugates with other chemical moieties, such as glycosyl groups, lipids, phosphate, acetyl groups and the like. Covalent derivatives of BNIP3 protein can be prepared by linking the chemical moieties to functional groups on amino acid side chains of the protein or at the N-terminus or at the C-terminus of the polypeptide.

### Animal Subjects, Target Tissues, Target Cells

The invention provides methods involving modulating levels of BNIP3 in a cell than can be in a target tissue that can be in an animal subject. Animal subjects include any mammal such as human beings, rats, mice, cats, dogs, goats, sheep, horses, monkeys, apes, rabbits, cattle, etc. The animal subject can be in any stage of development including adults, young animals, and neonates. Animal subjects also include those in a fetal stage of development. Target tissues can be any within the animal subject such as liver, kidney, heart (especially cardiomyocytes), lungs, components of gastrointestinal tract, pancreas, gall bladder, urinary bladder, skeletal muscle, the central nervous system including the brain, eye, skin, bones, etc.

### Viral Vectors

Various techniques using viral vectors for the introduction of a BNIP3 nucleic acid into a cell may be utilized in the methods of the invention. Preferred viral vectors for use in the invention are those that exhibit low toxicity to a host cell and induce production of therapeutically useful quantities of a BNIP3 protein or antisense nucleic acid in a tissue-specific manner. Viral vector methods and protocols that may be used in the invention are reviewed in Kay et al. Nature Medicine 7:33-40, 2001. The use of specific vectors, including those based on adenoviruses, adeno-associated viruses, herpes viruses, and retroviruses are described in more detail below.

The use of recombinant adenoviruses as gene therapy vectors is discussed in W. C. Russell, Journal of General Virology 81:2573-2604, 2000; and Bramson et al., Curr. Opin. Biotechnol. 6:590-595, 1995. Adenovirus vectors are preferred for use in the invention because they (1) are capable of highly efficient gene expression in target cells and (2) can accommodate a relatively large amount of heterologous (non-viral) DNA. A preferred form of recombinant adenovirus is a “gutless, “high-capacity”, or “helper-dependent” adenovirus vector. Such a vector features, for example, (1) the deletion of all or most viral-coding sequences (those sequences encoding viral proteins), (2) the viral inverted terminal repeats (ITRs) which are sequences required for viral DNA replication, (3) up to 28-32 kb of “exogenous” or “heterologous” sequences (e.g., sequences encoding a BNIP3 protein), and (4) the viral DNA packaging sequence which is required for packaging of the viral genomes into infectious capsids. For specifically cardiomyocytes, preferred variants of such recombinant adenoviral vectors contain tissue-specific (e.g., heart) enhancers and promoters operably linked to a BNIP3 gene.

Other viral vectors that might be used in the invention are adeno-associated virus (AAV)-based vectors. AAV-based vectors are advantageous because they exhibit high transduction efficiency of target cells and can integrate into the host genome in a site-specific manner. Use of recombinant AAV vectors is discussed in detail in Tal, J., J. Biomed. Sci. 7:279-291, 2000 and Monahan and Samulski, Gene Therapy 7:24-30, 2000. A preferred AAV vector comprises a pair of AAV inverted terminal repeats which flank at least one cassette containing a tissue (e.g., heart)- or cell (e.g., cardiomyocyte)-specific promoter operably linked to a BNIP3 nucleic acid. The DNA sequence of the AAV vector, including the ITRs, the promoter and BNIP3 gene may be integrated into the host genome.

The use of herpes simplex virus (HSV)-based vectors is discussed in detail in Cotter and Robertson, Curr. Opin. Mol. Ther. 1:633-644, 1999. HSV vectors deleted of one or more immediate early genes (IE) are advantageous because they are generally non-cytotoxic, persist in a state similar to latency in the host cell, and afford efficient host cell transduction. Recombinant HSV vectors can incorporate approximately 30 kb of heterologous nucleic acid. A preferred HSV vector is one that: (1) is engineered from HSV type I, (2) has its IE genes deleted, and (3) contains a tissue-specific (e.g., heart) promoter operably linked to a BNIP3 nucleic acid. HSV amplicon vectors may also be useful in various methods of the invention. Typically, HSV amplicon vectors are approximately 15 kb in length, and possess a viral origin of replication and packaging sequences.

Retroviruses such as C-type retroviruses and lentiviruses might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a BNIP3 nucleic acid. In methods of delivery to a heart, it may also encode a ligand to a cardiomyocyte-specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells. Lentiviral vectors for use in the invention may be derived from human and non-human (including SUV) lentiviruses. Preferred lentiviral vectors include nucleic acid sequences required for vector propagation as well as a tissue-specific promoter (e.g., heart) operably linked to a BNIP3 gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain (1) alphavirus genetic elements required for RNA replication, and (2) a heterologous nucleic acid such as one encoding a BNIP3 nucleic acid. Within an alphivirus replicon, the heterologous nucleic acid may be operably linked to a tissue-specific (e.g., heart) promoter or enhancer.

Recombinant, replication-defective alphavirus vectors are advantageous because they are capable of high-level heterologous (therapeutic) gene expression, and can infect a wide host cell range. Alphavirus replicons may be targeted to specific cell types (e.g., cardiomyocytes) by displaying on their virion surface a functional heterologous ligand or binding domain that would allow selective binding to target cells expressing a cognate binding partner. Alphavirus replicons may establish latency, and therefore long-term heterologous nucleic acid expression in a host cell. The replicons may also exhibit transient heterologous nucleic acid expression in the host cell. A preferred alphavirus vector or replicon is non-cytopathic.

In many of the viral vectors compatible with methods of the invention, more than one promoter can be included in the vector to allow more than one heterologous gene to be expressed by the vector.

To combine advantageous properties of two viral vector systems, hybrid viral vectors may be used to deliver a BNIP3 nucleic acid to a target tissue (e.g., heart). Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the host cell genome and effect stable BNIP3 gene expression.

### Non-Viral Delivery

In addition to viral vector-based methods, non-viral methods may also be used to introduce a BNIP3 nucleic acid into a host cell. A review of non-viral methods of gene delivery is provided in Nishikawa and Huang, Human Gene Ther. 12:861-870, 2001. A preferred non-viral gene delivery method according to the invention employs plasmid DNA to introduce a BNIP3 nucleic acid into a cell. Plasmid-based gene delivery methods are generally known in the art and are described in references such as Ilan, Y., Curr. Opin. Mol. Ther. 1:116-120, 1999, Wolff, J. A., Neuromuscular Disord. 7:314-318, 1997 and Arztl, Z., Fortbild Qualitatssich 92:681-683, 1998.

Methods involving physical techniques for introducing a BNIP3 nucleic acid into a host cell can be adapted for use in the present invention. For example, the particle bombardment method of gene transfer utilizes an Accell device (gene gun) to accelerate DNA-coated microscopic gold particles into target tissue, e.g., the liver. See, e.g., Yang et al., Mol. Med. Today 2:476-481 1996 and Davidson et al., Rev. Wound Repair Regen. 6:452-459, 2000. As another example, cell electropermeabilization (also termed cell electroporation) may be employed to deliver BNIP3 nucleic acids into cells. See, e.g., Preat, V., Ann. Pharm. Fr. 59:239-244 2001.

Synthetic gene transfer molecules can be designed to form multimolecular aggregates with plasmid DNA (e.g., harboring a BNIP3 coding sequence operably linked to a heart-specific promoter). These aggregates can be designed to bind to a target cell surface in a manner that triggers endocytosis and endosomal membrane disruption. Cationic amphiphiles, including lipopolyamines and cationic lipids, may be used to provide receptor-independent BNIP3 nucleic acid transfer into target cells. In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

DNA microencapsulation may be used to facilitate delivery of a BNIP3 nucleic acid. Microencapsulated gene delivery vehicles may be constructed from low viscosity polymer solutions that are forced to phase invert into fragmented spherical polymer particles when added to appropriate nonsolvents. Methods involving microparticles are discussed in Hsu et al., J. Drug Target 7:313-323, 1999 and Capan et al., Pharm. Res. 16:509-513, 1999.

Protein transduction offers an alternative to gene therapy for the delivery of therapeutic proteins into target cells, and methods involving protein transduction are within the scope of the invention. Protein transduction is the internalization of proteins into a host cell from the external environment. The internalization process relies on a protein or peptide which is able to penetrate the cell membrane. To confer this ability on a normally non-transducing protein, the non-transducing protein can be fused to a transduction-mediating protein such as the antennapedia peptide, the HIV TAT protein transduction domain, or the herpes simplex virus VP22 protein. See Ford et al., Gene Ther. 8:1-4, 2001.

### EXAMPLES

The present invention is further illustrated by the following specific examples. The examples are provided for illustration only and are not to be construed as limiting the scope or content of the invention in any way.

### Example 1

### Materials and Methods

Reagents. Antibodies to Bax, Bcl-XL, BAD, Bcl-2, and actin were from Santa Cruz Biotech (Santa Cruz, Calif.), anti-Bak was from LXR Biotechnology, (Richmond, Calif.), anti-BNIP3 was from BD Pharmingen (San Diego, Calif.). Anti-vertebrate sarcomeric myosin antibody (MF-20) was from the Developmental Studies Hybridbma Bank, University of Iowa. Caspase inhibitors Boc-D and ZVAD, Hoechst 33342, propidium iodide, trypan blue and anti-succinate dehydrogenase antibody were from CalBiochem (San Diego, Calif.). Plasmids BNIP3 and BNIP3delta-TM containing the wild type and transmembrane deleted BNIP3 cDNAs, respectively, were generous gifts from Don Dubik (University of Manitoba, Canada) (20). The green fluorescent protein (GFP) expression plasmid was from Cloneteck (Palo Alto, Calif.). BNIP3 antisense oligonucleotides containing phosphorothioate and fluorescein isothiocyanate tags were from Sigma Genosys (The Woodlands, TX). MitoTracker Red CMXRos was from Molecular Probes (Eugene Oreg.) All other reagents were from Sigma Chemical Co. (St. Louis, Miss.).

Cardiac Myocyte Culture and Hypoxia. Methods utilized for the isolation and culture of primary neonatal rat cardiac myocytes and exposure to hypoxia have been described previously (28;29). Experiments were performed in defined serum-free DMEM/M-199 (4:1) medium. Oxygen was continuously monitored and maintained at <10 mm Hg.

Quantitative Analysis of Apoptosis. Cells were examined for morphologic evidence of apoptosis or necrosis by staining with anti-myosin antibody and the fluorescent DNA-binding dyes Hoechst 33342 and propidium iodide (PI), exactly as previously described (26;30). Genomic DNA fragmentation analyses were also as described previously (26;30).

Northern and Western Blots. Northern and Western blot procedures were exactly as described previously (31). Northern blots were probed with full-length rat BNIP3 and β-actin icDNAs as indicated. Western blots were stained with Ponceau Red to monitor the transfer of proteins.

Antisense. Antisense oligonucleotides were complementary to bases 10-40 and 600-630 of the BNIP3 gene; sequences 5′ACGGGGACGATGGAGAGCCACTGGCGGAGG (SEQ ID NO:1) and 5′CCTAGATGTAACCTTCCGCAGACTGTTGAA (SEQ ID NO:2) respectively. Random sequence oligonucleotides contained the same bases in a scrambled sequence. FITC-tagged oligonucleotides (2 μM final of each) in DMEM were mixed with lipofectamine (0.1 μg/ml, final concentration) and incubated with cardiac myocytes at 37° C. for 8 hours before treatments. Immediately before exposing to hypoxia, fresh medium with oligonucleotides was added. Fluorescence was visualized after 24, 36, and 48 hrs of exposure to hypoxia. Fresh oligonucleotides (400 nM) were added at 24 and 36 hrs.

Necrosis Assays. Trypan blue exclusion assays were performed to identify compromised plasma membranes. Culture media was removed and replaced with 0.4% trypan blue in PBS for 15 min at 37° C.; positive cells were quantitated microscopically. Lactate dehydrogenase (LDH) in culture media was measured using a colorimetric LDH assay kit (Sigma, St. Louis, Mo.).

Subcellular Fractionation. Cell fractionation and alkali treatments were performed as described in (19). Briefly, following treatments, cells were washed with PBS and lysed by homogenizing in buffer containing 100 MM mannitol, 10 mM Tris, 5 mM MgCl₂, 1 mM EGTA, 1 mM DTT. Samples were split in two and the pH of one half was adjusted to 11.0 with 0.1 M NaHCO₃ and incubated on ice for 20 min. Samples were fractionated by differential centrifugation. Intact cells and nuclei were separated by centrifugation at 120 g for 5 minutes; supernatants were centrifuged at 10,000 g for 10 minutes to collect the heavy (mitochondrial) membrane pellet. Cytoplasmic fractions were obtained by centrifuging supernatants at 100,000 g for 30 minutes.

MitoTracker Red labeling and confocal microscopy. Cardiac myocytes plated on Nunc glass dishes were incubated with 0.2 μM MitoTracker Red, in serum-free media for 20 minutes. Cells were rinsed and fixed for 30 min with 3.7% paraformaldehyde in PBS. To analyze BNIP3-transfected cardiac myocytes, cultures were cotransfected with pGFP to identify transfected cells and stained as above. Cells were analyzed using an Olympus 1X70 inverted confocal laser microscope at excitation wavelength 579 nm and emission wavelength 599 nm; GFP was visualized at 520 nm.

Transfections. Cardiac myocytes were transfected on day 1 after isolation using polycationic liposomes as described previously (30). Transfection efficiency was 11.5±1.6% as estimated by GFP expression from a transfected GFP plasmid (32). The transfection procedure alone did not affect the level of apoptosis during treatments (data not shown). Apoptosis of transfected myocytes was quantitated by cotransfecting the β-Gal gene, co-staining with X-gal and Hoechst 33342, and counting condensed Hoechst-positive nuclei as described previously (30). For acidosis, the pH of the medium was adjusted to 6.5 by adding lactic acid (16.5 mM) and phosphoric acid, as described previously (26). The pH was maintained at 6.5 for the duration of the incubation by adding additional phosphoric acid as necessary.

Statistics. Error bars represent SEM; significance was calculated using ANOVA software.

### Example 2

### Results

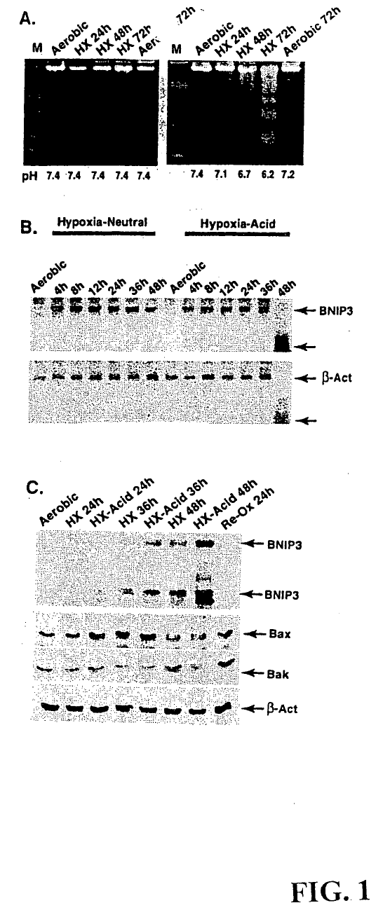

BNIP3 accumulation and cell death during hypoxia-acidosis. DNA fragmentation and nuclear condensation were measured in cardiac myocytes subjected to hypoxia in neutral or acidic pH media. Extensive fragmentation of DNA was observed in hypoxic acidotic cells, but not in hypoxic cells maintained at neutral pH (FIG. 1A). After 72 h of hypoxia without neutralization, the [pH]o fell to 6.4, and 63+8% of myocytes contained condensed Hoechst and TUNEL-positive nuclei, compared with 7.1% of cells in pH-neutral cultures. There were no significant changes in PI staining under either condition. BNIP3 accumulation is shown in FIGS. 1 (B and C). BNIP3 mRNA levels increased progressively during hypoxia and peaked after 8 h at similar levels in both neutralized and acidic conditions. BNIP3 mRNA was degraded after 48 h of hypoxia in acidic but not in neutral pH media in parallel with cell death. BNIP3 protein accumulated more rapidly under acid pH and peaked at a significantly higher level than the pH-neutral samples (3.3±0.7-fold; n=3; p<0.01). There were no corresponding changes in Bax, Bak or β-actin proteins; the apparent small increase of Bax at 24 and 36 h was not reproducible (see ref (27)). These results demonstrate that hypoxia activates BNIP3 transcription and protein accumulation is stabilized by acidosis.

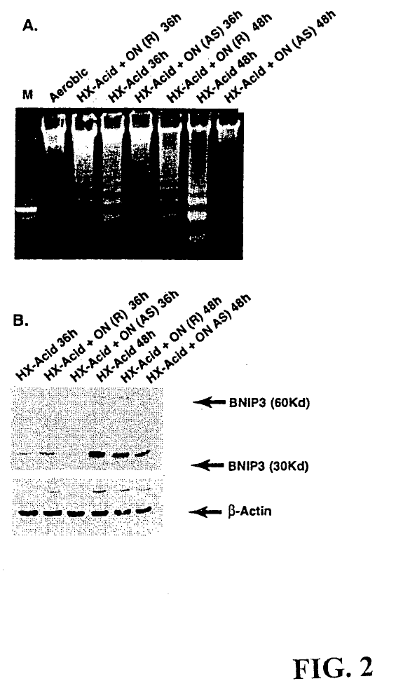

Cell death is blocked by BNIP3 antisense oligonucleotides. To determine if there was a relationship between BNIP3 accumulation and cell death, cardiac myocytes were treated with antisense BNIP3 oligonucleotides before and during exposure to hypoxia as described in Methods. As shown in FIG. 2A, treatment with antisense BNIP3 reduced DNA fragmentation markedly at both time points. Random sequence oligonucleotides also delayed DNA fragmentation slightly, reflecting a non-specific side effect of oligonucleotide treatment. Uptake of oligonucleotides was >95%, estimated microscopically by visualizing the fluorescent tag. Antisense oligonucleotides reduced BNIP3 protein by 78+8% (n=3) during the incubations. There was no significant effect of oligonucleotide treatment on levels of β-actin.

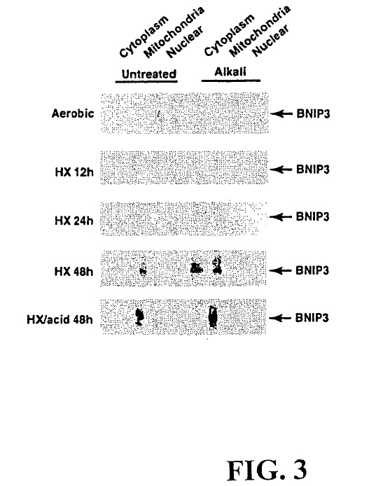

Acidosis increases BNIP3 binding to mitochondrial membranes. BNIP3 accumulates under hypoxia at neutral and acidic pH but cell death occurs only with coincident acidosis. To test the possibility that low pH stimulates intracellular translocation of BNIP3, cells were separated into subcellular fractions after hypoxia exposure. After cell lysis, samples were treated with alkaline buffer to dislodge loosely membrane-associated protein as described previously (19;21) Results from Western blots of untreated and alkali-solubilized fractions are shown in FIG. 3. BNIP3 levels were initially detected in the alkalinized cytoplasmic fraction at 12 h of hypoxia and increased progressively at 24 h and 48 h. BNIP3 was present exclusively in the mitochondrial fraction of hypoxic samples without alkali treatment, and was primarily mitochondrial in hypoxia-acidosis samples. Alkaline treatment caused a significant shift of BNIP3 into the cytoplasmic fraction from hypoxic-neutral but not hypoxia-acidic treatments. In 48 h-hypoxia-neutral samples 73% of BNIP3 was in the cytoplasm compared with <10% of the hypoxia-acidosis sample (mean of 2 determinations). Alkali treatment did not effect the distribution of succinate dehydrogenase that was localized in the mitochondrial and nuclear fractions in all samples. These results show that acidic pH promotes a stronger alkali-resistant association of BNIP3 with mitochondrial membranes.

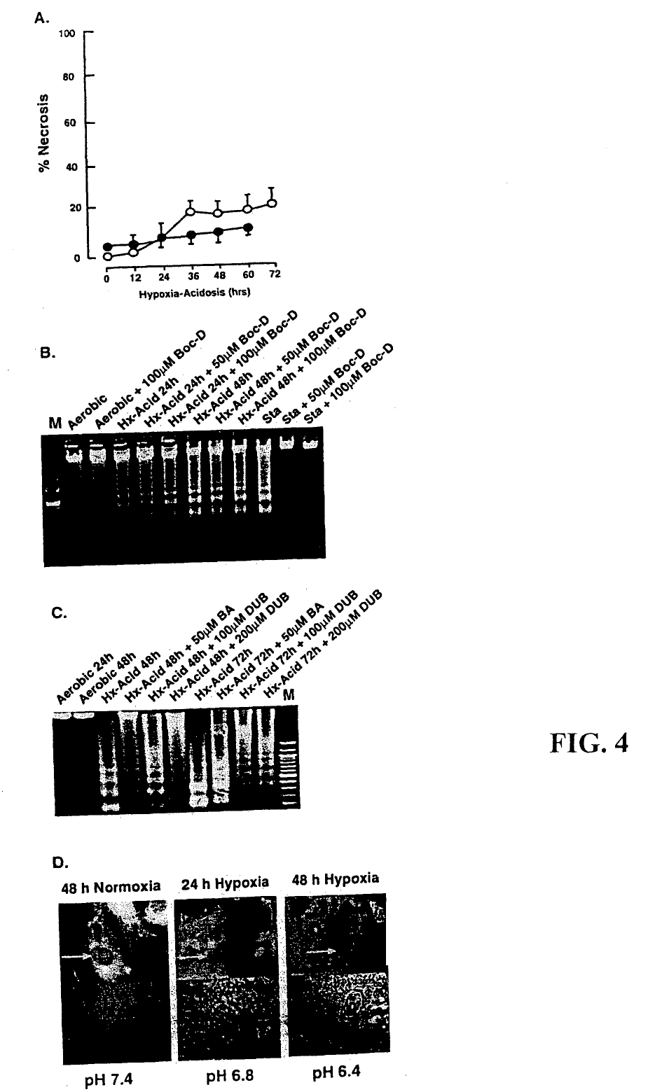

Characteristics of hypoxia-acidosis mediated death pathway. Overexpression of BNIP3 by transient transfection of cell lines was reported to activate a necrosis-like pathway that included early loss of plasma membrane integrity (19). To determine whether a similar pathway was activated by hypoxia-acidosis membrane permeability changes, caspase activity, and MPTP function were analyzed. Progressive hypoxia-acidosis with >70% cell death caused <20% loss of membrane integrity even at the late time points as determined by Trypan blue exclusion or LDH release (FIG. 4A). These results agree with a previous report that PI staining did not change significantly under these treatments (26). FIG. 4 panel B shows the effects of the broad range caspase inhibitor Boc-D on DNA fragmentation. DNA fragmentation was unaffected by Boc-D and the same result was obtained using another broad-range caspase inhibitor, ZVAD (not shown). In other experiments, no cleavage of the caspase-3 substrate PARP was detected in extracts of hypoxia-acidosis treated cells.

To test for a contribution of MPTP activity in this pathway, cardiac myocytes were exposed to hypoxia-acidosis in the presence and absence of the specific MPTP inhibitors bongrekic acid (BA) and decylubiquinone (DUB). Fragmentation of cardiac myocyte DNA by hypoxia-acidosis was blocked at the 48 h time point by either BA or 200 μM DUB (FIG. 4C, lanes 4 and 6). Even after 72 h when the control DNA was fully cleaved into small fragments, both BA and DUB treatment mediated protection. As an additional test for MPTP opening, cardiac myocytes were loaded under identical conditions with MitoTracker Red dye and analyzed as described in Methods. Aerobic cultures displayed bright punctate patterns of intense staining around the nuclei, characteristic of mitochondria staining. Fluorescence was weaker in cells exposed to 24 h of hypoxia and absent after 48 h of hypoxia-acidosis (FIG. 4D, right panel). These results are consistent with hypoxia-acidosis induced MPTP opening as part of the death pathway. Significant leakage of cytochrome c from the mitochondria during hypoxia-acidosis was not detected under conditions where significant leakage occurred from GSNO-treated myocytes (32). This result seems anomalous with MPTP opening but is consistent with a previous report documenting a similar effect on BNIP3-transfected cells (19).

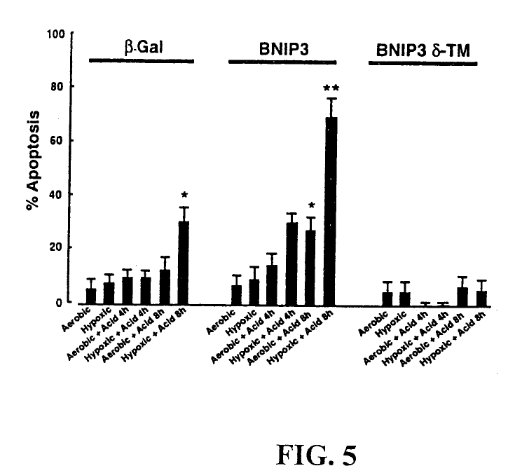

Acid and hypoxia-dependent death induced by wild type BNIP3 transfection. These results indicate that the accumulation of endogenous BNIP3 protein during hypoxia is not sufficient to activate cardiac myocyte death at neutral pH. To determine whether transfected BNIP3 is also regulated by pH, cardiac myocytes were transfected with wild type BNIP3, BNIP3 with a deletion in the TM domain (BNIP3delta-TM) or empty vector. Cells were co-transfected with β-gal to identify transfected cells, and co-stained with sarcomeric myosin to identify myocytes. Apoptotic nuclei were quantitated as described previously (26;30). After transfection, cultures were exposed to aerobic or hypoxia conditions at low or neutral pH for the time periods indicated in FIG. 5. Acidic media, with or without hypoxia, caused a small increase of the apoptotic index in control cells at 4 h and 8 h but the increase was only significant after 8 h of hypoxia-acidosis. Apoptotic indices of BNIP3-transfected cells were not significantly different from controls under aerobic, hypoxic or aerobic-acidotic 4 h treatments. However, the indices were significantly increased in 4 h hypoxia-acidotic and both 8 h treatments compared to aerobic or hypoxia only treatments and controls. The significant increase of apoptosis by acid treatment under aerobic incubation confirms the regulation of BNIP3 activity by pH. The synergistic effect of combined acid and hypoxia probably reflects the lower intracellular pH mediated by this condition. BNIP3delta-TM-transfected myocytes exhibited lower rates of apoptosis than both BNIP3 and control. The lower incidence of apoptosis was significantly different from the control under the conditions of 8 h hypoxia with acidosis. This may reflect a dominant negative effect of BNIP3delta-TM.

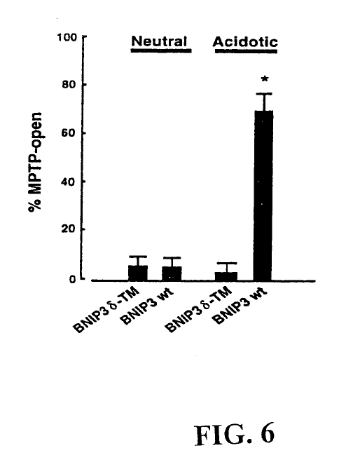

Wild type BNIP3 transfection stimulates MPTP opening. To determine whether MPTP opening was associated with BNIP3 activity, cardiac myocytes were co-transfected with pGFP and either wild type BNIP3 or BNIP3delta-TM and exposed to neutral or acidic pH for 8-h. Cultures were stained with MitoTracker Red, fixed and analyzed by confocal microscopy. GFP-positive cells were scored as MPTP-closed if the staining was sharp and punctate as shown in FIG. 4. In untreated-transfected cultures 95% of GFP-positive cells were scored as closed-MPTP; this fraction did not change significantly in the BNIP3delta-TM-acid treatment group. In contrast, there was a significant increase of open-MPTP myocytes in cultures transfected with wild type BNIP3 and subjected to acidic conditions (FIG. 6). These results confirm that MPTP opening is an integral part of this pathway of cardiac myocyte death mediated by BNIP3 and acidosis.

### Example 3

### Discussion

BNIP3 mRNA and protein were almost undetectable in aerobic myocytes but accumulated significantly during hypoxia. Acidosis enhanced BNIP3 protein, but not mRNA, accumulation by almost 3-fold, indicating increased protein translation or stability at low pH. Antisense-mediated depletion of BNIP3 dramatically reduced cardiac myocyte death by hypoxia-acidosis. Despite significant accumulation of BNIP3 under hypoxia at neutral pH, cell death did not occur without coincident acidosis. This suggests that at neutral pH, BNIP3 exists in an inactive state, possibly similar to other Bcl-2 family proteins that require a death signal for activation (22).

Acidosis promoted a tighter association between BNIP3 and the mitochondria, since BNIP3 could be dislodged from hypoxia-neutral myocyte mitochondria by alkali treatment, but not from hypoxia-acidotic mitochondria. Therefore integration of BNIP3 into mitochondrial membranes, accelerated by acidosis may constitute the activation step (21). Acid-mediated stabilization of BNIP3 may reflect this sequestration by protecting against cellular proteases. Alternatively the increased myocyte death during acidosis may simply reflect enhanced levels of BNIP3 tilting the balance between pro- and anti-apoptotic Bcl-2 proteins.

The death pathway mediated by BNIP3 is unusual in that it does not appear to involve caspase activation. Cell death was not blocked by either of two broad range caspase inhibitors, and PARP, a caspase-3 substrate, did not undergo detectable cleavage (26). However, MPTP opening appears to be part of the BNIP3-mediated program in the system examined here. MPTP inhibitors effectively prevented cell death, and MitoTrack Red dye was not retained in cells subjected to hypoxia-acidosis or in BNIP3-transfected cells subjected to acidosis. The loss of MitoTrack dye suggests that BNIP3 integration mediates increased mitochondrial permeability and probably loss of membrane potential.

Analyses of BNIP3-transfected cardiac myocytes confirmed that pH regulates the function of this protein. Exposure of wild type BNIP3-transfected myocytes to acid caused a 4-6-fold increase in the apoptotic index compared with pH-neutral, aerobic cells; the combination of acidosis and hypoxia caused a 12-fold increase. Both values were significantly different from non-acidic aerobic or hypoxic BNIP3-transfected cells, or from control, empty vector-transfected cells. This provides strong supporting evidence that both endogenous and transfected BNIP3 are subject to pH regulation. Interestingly, the apoptotic index of cardiac myocytes transfected with BNIP3delta-TM was significantly lower than that in cells transfected with either wild type BNIP3 or empty vector after treatments. A possible explanation for this is that BNIP3delta-TM dimerizes with endogenously generated BNIP3 and the heterodimers are unable to integrate into the mitochondrial membranes. Therefore BNIP3delta-TM behaves like a dominant negative.

Although the experiments described herein were implemented using neonatal cardiac myocytes, BNIP3 mRNA and protein was also detected in intact adult hearts and it seems probable that a similar death pathway occurs in other cell types including those in ischemic heart tissue.

### Example 4

### Peptide Fragments of BNIP3 Protect Against Hypoxia-Acidosis

Peptides containing N-terminal amino acids 1-23 and 24-49 with TAT sequences as indicated below to mediate transport of the peptide into cardiac myocytes were synthesized.

Peptide 1 (SEQ ID NO:3)
GRKKRRQRRRPPQC-----
MSQSGEENLQGSWVELHFSNGNG-(C-term)
Peptide 2 (SEQ ID NO:4)
2.GRKKRRQRRRPPQC----
GDMEKILLDAQHESGRSSKSSHCDSP-(C-term)

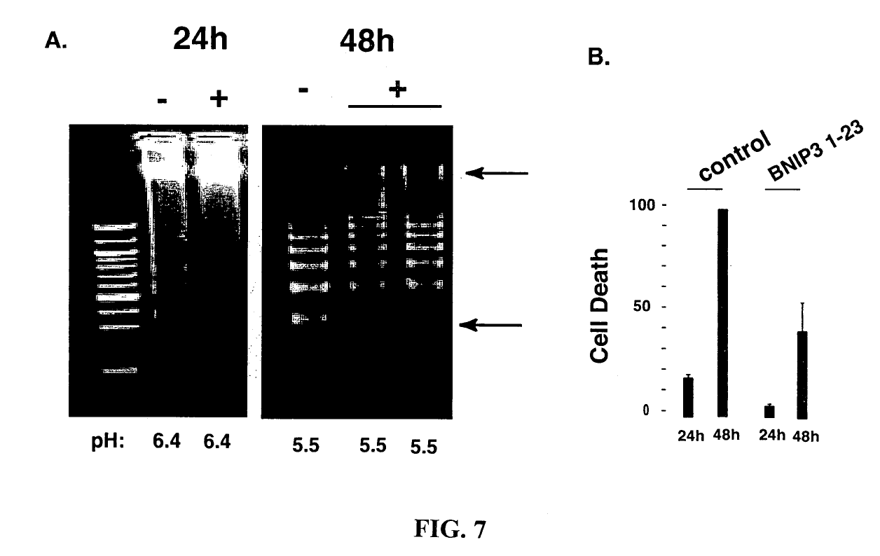

Peptides were added to cardiac myocyte cultures at a concentration of 10 PM (20 μg/ml) in serum-free defined medium (DMEM/high glucose/TIB) as described previously (26). Control cultures contained 20 μg/ml BSA. Cultures were exposed to 24-48 h of hypoxia with acid accumulation, also as described previously (26). The results using peptide 1 (23 aa N-BNIP3) are shown in FIG. 7 (−is control +is with peptide treatment). As indicated Peptide 1-treatment mediated a strong and significant protection against hypoxia-acidosis death. Arrows in FIG. 7A show high molecular weight genomic DNA was only present at 48 h in the peptide treatment group and this group did not contain any low molecular size (fully degraded) DNA (bottom arrow). Quantitative DNA fragmentation analyses (see FIG. 7B) indicated that peptide 1 conferred >50% protection at both 24 h and 48 time points (n=3). Peptide 2 was less effective (non-BNIP3-specific peptides were neutral, data not shown). These data indicate that peptides derived from the BNIP3 sequence can protect cardiac myocytes against death mediated by hypoxia-acidosis.

### REFERENCES

1. Jennings, R. B., Steebergen, C., & Reimer, K. A. (1995) Monogr.Pathol. 37, 47-80.

2. Vanoverschelde, J-L J., Wijns, W., Borgers, M., Heyndrickx, G., Depre, C., Flambeng, W., and Melin, J. A. (1997) Circulation 95, 1961-1971.

3. Zuurbier, C. J., van Iterson, M., & Ince, C. Card. Res. (1999) 44 (3), 488-497.

4. Narula, J., Hajjar, R. J., & Dec, G. W. (1998) Cardiology Clinics 16 (4), 691-710.

5. Dennis, S. C., Gevers, W., & Opie, L. H. (1991) J.Mol.Cell Cardiol. 23, 1077-1086.

6. Neely, J. R. & Grotyohann, L. W. (1984) Circ.Res. 55, 816-824.

7. Webster K. A, Discher D J, Hernandez O M, Yamashita K, & Bishopric N H. (2000) Adv Exp Med Biol. 475, 161-175.

8. Kajstura, J., Cheng, W., Reiss, K., & Clark, W. A. (1996) Lab.Invest. 74, 86-107.

9. Gottlieb, R. A., Burleson, K. O., Kloner, R. A., Babior, B. M., & Engler, R. L. (1994) J.Clin.Invest. 94, 1612-1628.

10. Anversa, P. & Kajstura, J. (1998) Circ.Res. 82, 1231-1233.

11. Horwitz, L. D., Fennessey, P. V., Shikes, R. H., & Kong, Y. (1994) Circulation. 89, 1792-1801.

12. Yoshida T., Watanabe M., Engelman D. T., Engelman R. M., Schley J. A., Maulik N., Ho Y. S., Oberley T. D., & Das D. K. (1996) J Mol Cell Cardiol 28, 1759-67

13. Fliss, H. & Gattinger, D. (1996) Circ.Res. 79, 949-956.

14. Williams, F. M., Kus, M., Tanda, K., & Wlliams, T. J. (1994) Br.J.Pharmac. 111, 1123-1128.

15. Hochachka, P. W., Buck, L. T., Doll, C. J., & Land, S. C. (1996) Proc.Natl.Acad.Sci. USA 93, 9493-9498.

16. Buja, L. M., Eigenbrodt, M. L., & Eigenbrodt, E. H. (1993) Arch.Pathol.Lab.Med. 117, 1208-1214.

17. Majno, G. & Joris, I. (1995) Am.J.Pathol. 146, 3-15.

18. Reimer, K. A. & Ideker, R. E. (1987) Human Pathol. 18, 462-475.

19. Vande Velde C, Cizeau J, Dubik D, Alimonti J, Brown T, Israels S, Hakem R, & Greenberg A H. (2000) Mol Cell Biol. 20(15), 5454-68.

20. Ray R, Chen G, Vande Velde C, Cizeau J, P. J., Reed J C, Gietz R D, & Greenberg A H (2000) J Biol Chem. 2 275, 1439-48.

21. Goping, I. S., Gross, A., Lavoie, J. N., Nguyen, M., Jemmerson, R., Roth, K., Korsemeyer, S. J., and Shore, G. C. J. Cell Biol. (1998)143, 207-215.

22. Adams, J. M. and Cory, S. Trends Biochem. Sci. 26(1), 61-66. 2001.

23. Green, D. & Reed, J. (1998) Science 281, 1309-14.

24. Earshaw, W., Martins, L., & Kaufmann, S. (1999) Annu. Rev. Biochem. 68, 383-424.

25. Crompton M. (2000) Curr Opin Cell Biol 12, 414-9.

26. Webster, K. A., Discher, D). J., Kaiser, S., Hernandez, 0. M., Sato, B., & Bishopric, N. H. (1999) J.Clin.Invest. 104, 239-252.

27. Karwatowska-Prokopczuk, E., Nordberg, J. A., Li, H. L., Engler, R. L., & Gottlieb, R. A. (1998) Circ.Res. 82, 1139-1144.

28. Webster, K. A. & Bishopric, N. H. (1992) J.Mol.Cell.Cardiol. 24, 741-751.

29. Webster, K. A., Discher, D., & Bishopric, N. H. (1993) J.Biol.Chem. 268, 16852-16859.

30. Dougherty, C. J., Kubasiak, L., Prentice, H., Andreka, P., Bishopric, N. H., and Webster, K. A. (2002) Biochemical Journal 362, 561-571.

31. Webster, K. A., Muscat, G. E. O., & Kedes, L. (1988) Nature 332, 553-561.

32. Andreka P, Zang J, Dougherty C, Slepak T I, Webster K A, & Bishopric N H. (2001) Circ. Res. 88, 305-312.

33. Webster, K. A., Discher, D. J., & Bishopric, N. H. (1994) Circ.Res. 75, 361-371.

34. Gottlieb, R. A., Giesing, H. A., Zhu, J. Y., Engler, R. L., & Babior, B. M. (1995) Proc.Natl.Acad.Sci. USA 92, 5965-5968.

35. Perez-Sala, D., Collado-Escobar, D., & Mollinedo, F. (1998) J. Biol. Chem. 270, 6235-6242.

36. Kajstura, J., Leri, A., Finato, N., Loreto, C., Beltrami, C. A., & Anversa, P. (1998) Proc.Natl.Acad.Sci. USA 95, 8801-8805.

37. Narula, J., Haider, N., Virmani, R., DiSalvo, T. G., Kolodgie, F. D., Hajjar, R. J., Schmidt, Y., Semigran, M. J., Dec, G. W. et al (1996) N.Engl.J.Med. 1182, 1182-1189.

38. Heusch, G. and Schulz, R. (1996) Hibernating myocardium: a review. J. Mol. Cell Cardiol. 28(12), 2359-2372.

39. Chen, C., Ma, L., Linfert, D. R., Lai, T., Fallon, J. T., Gillam, L. D., Waters, D. D., & Tsongalis, G. J. (1997) J.Am.Coll.Card. 30, 1407-1412.

40. Kajstura, J., Cheng, W., Reiss, K., Clark, W. A., Sonnenblick, E. H., Krajewski, S., Reed, J. C., Olivetti, G., & Anversa, P. (1996) Lab Invest. 74, 86-107.

41. Ohno, M., Takemura, G., Ohno, A., Misao, J., Hayakawa, Y., Minatoguchi, S., Fujiwara, T., & Fujiwara, H. (1998) Circulation 98, 1422-1430.

42. Krayenbuehl, H. P. & Hess, 0. M. (1992) Journal of Myocardial Ischemia. 4, 49-58.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A non-naturally occurring method for preventing or reducing hypoxia-acidosis induced injury to a cell, the method comprising the step of: reducing BNIP3 expression or activity in the cell.

2. The method of claim 1, wherein the step of reducing BNIP3 expression or activity in the cell comprises decreasing the amount of BNIP3 mRNA in the cell.

3. The method of claim 1, wherein the step of reducing BNIP3 expression or activity in the cell comprises decreasing the amount of BNIP3 protein in the cell.

4. The method of claim 1, wherein the step of reducing BNIP3 expression or activity in the cell comprises introducing an antisense oligonucleotide into the cell.

5. The method of claim 1, wherein the step of reducing BNIP3 expression or activity in the cell comprises expressing a mutant BNIP3 protein in the cell.

6. The method of claim 1, wherein the step of reducing BNIP3 expression or activity in the cell comprises preventing BNIP3 protein dimerization in the cell.

7. The method of claim 1, wherein the step of reducing BNIP3 expression or activity in the cell comprises preventing translocation of BNIP3 protein to a mitochondrion in the cell.

8. The method of claim 1, wherein the step of reducing BNIP3 expression or activity in the cell comprises preventing or reversing acidosis in the cell.

9. The method of claim 1, wherein the cell is a myocyte.

10. The method of claim 9, wherein the cell is a cardiomyocyte.


SEQUENCE LISTING (4 sequences)

SEQ ID NO: 1 - DNA, 30 bp, Artificial
  Antisense Oligonucleotide
acggggacgatggagagccactggcggagg

SEQ ID NO: 2 - DNA, 30 bp, Artificial
  Antisense Oligonucleotide
cctagatgtaaccttccgcagactgttgaa

SEQ ID NO: 3 - protein, 37 aa, Artificial
  Synthetic Peptide Fragments
GRKKRRQRRRPPQCMSQSGEENLQGSWVELHFSNGNG

SEQ ID NO: 4 - protein, 40 aa, Artificial
  Synthetic Peptide Fragments
GRKKRRQRRRPPQCGDMEKILLDAQHESGRSSKSSHCDSP